(19) United States
(12) Patent Application Publication
(10) Pub. No.: US 2025/0170219 A1
(43) Pub. Date: May 29, 2025

(54) Fast Acting Joint Health Composition and Use Thereof

(71) Applicant: Lonza Greenwood LLC, Greenwood, SC (US)

(72) Inventors: Vijaya Juturu, North Brunswick, NJ (US); Jowell Bolivar, Greenwood, SC (US); Shane Durkee, Greenwood, SC (US); Tyler White, Greenwood, SC (US); Zainulabedin Saiyed, Benicia, CA (US)

(21) Appl. No.: 18/833,977

(22) PCT Filed: Jan. 31, 2023
(86) PCT No.: PCT/US2023/011992

§ 371 Date: Jul. 29, 2024

Related U.S. Application Data
(60) Provisional application No. 63/305,909, filed on Feb. 2, 2022.

(51) Int. Cl. A61K 38/39; A61K 9/16; A61K 31/365; A61K 31/56; A61K 36/48

(52) U.S. Cl. CPC A61K 38/39; A61K 9/1617; A61K 31/365; A61K 31/56; A61K 36/48

(57) ABSTRACT

A joint health composition is disclosed that, when administered to mammals in a therapeutically effective amount, can reduce inflammation and joint pain. The joint health composition contains a collagen, and at least one anti-inflammatory adjuvant in the delivery form of lipid multiparticulates. The joint health composition show increased bioavailability and a reduction in inflammation and joint pain in a reduced time period.

### CROSS REFERENCE TO RELATED APPLICATIONS

This application is based on and claims priority to U.S. Provisional Application No. 63/305,909, filed on Feb. 2, 2022, which is expressly incorporated herein by reference in its entirety.

### BACKGROUND

The matrix of cartilage is comprised of collagens, proteoglycans, and non-collagenous proteins and serves as the cushion and shock absorber within skeletal articulations as it lines the ends of the two bones that form the joint. Cartilage damage can be caused by several conditions including: joint injury, avascular necrosis, the effects of aging, osteoarthritis, auto immune disorder, and rheumatoid arthritis. The damaged cartilage causes pain and can limit the motion of the joint. For instance, osteoarthritis (OA) includes progressive loss of articular cartilage, subchondral bone sclerosis, and synovial inflammation, and is now the most common chronic condition worldwide.

Inflammation is a response of a tissue to injury and is characterized by increased blood flow to the tissue causing increased temperature, redness, swelling, and pain. Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

Joint health factors that indicate the onset of poor joint health caused by injury or osteoarthritis include interleukin-1 (IL-1), interlukin-6 (IL-6), and tumor necrosis factor alpha (TNF α). TNFα, IL-1, and IL-6 are primarily produced by macrophages. TNF α exists as both a soluble form, solTNF, which is believed to play an important role in inflammation, and a transmembrane form, tmTNF, which is involved in immune functions. Interleukin-1 is a family of 11 cytokines that are regulators of inflammation. Similarly, interleukin-6 is pro-inflammatory cytokine that is produced in response to tissue injuries.

TNFα, IL-1, and IL-6 exerts primary effects on monocytes, synovial macrophages, fibroblasts, chondrocytes, and endothelial cells, and induce proinflammatory cytokine and chemokine synthesis. TNFα, IL-1, and IL-6 also promotes secretion of matrix metalloproteinases (MMPs), leading to cartilage matrix degradation, which indicates inflammation.

Because it initiates an inflammatory cascade and has been found to be increased in close proximity to inflamed or injured tissue, TNFα, IL-1, and/or IL-6 inhibition is a target for pain and/or inflammation therapy and/or tissue destruction. Furthermore, alone or in conjunction with any of the above noted inflammation biomarkers, inflammation and poor joint health contribute to a perception of pain in the affected mammal. This subjective pain is often what is measured or reported to pain management professionals, even when one or more of the biomarkers above may also be present.

Various methods exist for treating skeletal articulations that suffer from injury or disease, such as osteoarthritis. Most of these treatment methods are directed to controlling pain and inflammation. However, existing treatment methods and compositions fail to provide relief in a timely manner, such as about two weeks or less. Furthermore, existing composition require large dosages over time, as existing treatments have low bioavailability and bioactive nutrient efficacy.

Therefore, in one aspect, it would be a benefit to provide a joint health composition that provides relief from joint pain and/or joint inflammation in about 14 days or less. Furthermore, in an aspect, it would be a benefit to provide a joint health composition that has improved bioavailability and/or bioactive nutrient efficacy. It would further be a benefit to provide a joint health composition that reduces pro-cytokines, in one aspect, such as pain and inflammation associated with osteoarthritis. Moreover, it would be a benefit to provide a composition that has enhanced efficacy and absorption, even at reduced dosages and/or administration time periods.

### SUMMARY

The present disclosure is generally directed to a method of improving one or more of joint pain and inflammation in mammals. The method includes supplying a joint health supplement that includes a lipid multiparticulate and conjunction with collagen to the mammal, where the lipid multiparticulate includes at least one anti-inflammatory adjuvant. The anti-inflammatory adjuvant is an extract or isolate from a natural source that inhibits one or more of: interleukin-1 expression, interleukin-6 expression, NF-KB expression, cyclooxygenase-2 (COX-2) expression, inducible nitric oxide synthase (iNOS) expression, 5-lipoxygenase biosynthesis, and tumor necrosis factor-α (TNF-α) expression, Furthermore, in one aspect, the improvement in at least one of joint pain and inflammation is evidenced by a 10% or greater decrease in serum interleukin-1, interleukin-6, TNF-α, COX-2, SOX-9, MMP-13, or a combination thereof, a subjective reduction in one or more of joint pain and inflammation, or a combination thereof in about two weeks or less.

The present disclosure is also generally directed to joint health composition for improving one or more of joint health and inflammation. The joint health composition includes collagen and a lipid multiparticulate, therein the lipid multiparticulate contains at least one anti-inflammatory adjuvant. The anti-inflammatory adjuvant includes an extract or isolate from a natural source that inhibits one or more of: interleukin-1 expression, interleukin-6 expression, NF-KB expression, cyclooxygenase-2 (COX-2) expression, inducible nitric oxide synthase (iNOS) expression, 5-lipoxygenase biosynthesis, and tumor necrosis factor-α (TNF-α) expression. Moreover, in one aspect the composition improves one or more of joint pain and inflammation evidenced by a 10% or greater decrease in serum interleukin-1, interleukin-6, TNF-α, COX-2, SOX-9, MMP-13, or a combination thereof, a subjective reduction in one or more of joint pain and inflammation, or a combination thereof after administration of the composition to a mammal for about two weeks or less.

Furthermore, in one aspect, the present disclosure is also generally directed to a joint health composition for improving one or more of joint health and inflammation, that includes an extract and/or isolate obtained from Andrographis paniculate, Tamarind seed, green lipped mussel (Perna canaliculus), turmeric (Curcuma longa), stinging nettle (Urtica dioica), Cat's claw (Uncaria tomentosa), and bromelain, and collagen. In one aspect, the extract and/or isolate is from Andrographis paniculate, or Tamarind seed and the collagen is undenatured collagen.

Other features and aspects of the present disclosure are discussed in greater detail below.

### DEFINITIONS

As used in this application and in the claims, the singular forms “a,” “an,” and “the” include the plural forms unless the context clearly dictates otherwise. Additionally, the term “includes” means “comprises.” The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

Unless otherwise indicated, all numbers expressing quantities of ingredients, properties such as molecular weight, percentages, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited. As used herein, the terms “about,” “approximately,” or “generally,” when used to modify a value, indicates that the value can be raised or lowered by 10%, such as, such as 7.5%, 5%, such as 4%, such as 3%, such as 2%, such as 1%, and remain within the disclosed aspect. Moreover, the term “substantially free of” when used to describe the amount of substance in a material is not to be limited to entirely or completely free of and may correspond to a lack of any appreciable or detectable amount of the recited substance in the material. Thus, e.g., a material is “substantially free of” a substance when the amount of the substance in the material is less than the precision of an industry-accepted instrument or test for measuring the amount of the substance in the material. In certain example embodiments, a material may be “substantially free of” a substance when the amount of the substance in the material is less than 10%, less than 9%, less than 8%, less than 7%, less than 6%, less than 5%, less than 4%, less than 3%, less than 2%, less than 1%, less than 0.5%, or less than 0.1% by weight of the material.

The term “therapeutically effective amount” as used herein, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation. In one aspect, a therapeutically effective amount refers to supplying or ingesting collagen in an amount to maintain, repair, or improve the health of a joint(s). Furthermore, in one aspect, effectiveness as used herein can be a mammal's subjective measurement of an improvement in one or more of inflammation and joint pain.

The term “supplement” means a product in addition to the normal diet of the mammal but may be combined with a mammal's normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

The term “nutraceutical” and refers to any compound added to a dietary source (e.g., a food, beverage, or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “mammal” includes any mammal that may benefit from improved joint health, resilience, and recovery, and can include without limitation canine, equine, feline, bovine, ovine, human, or porcine mammals.

As used herein, “healthy” refers to the absence of illness or injury.

Unless otherwise noted, “collagen” as used herein refers to all forms of collagen, either with or without denaturation, without or without salts or stabilizing agents, and fibrillar and non-fibrillar types of collagen not limited to fibril associated collagens with interrupted triple helices (FACIT, Type IX, XII, XIV, XIX, XXI), including short chain collagen (generally Types VII and X), basement membrane (Type IV), Multiplexin (multiple triple helix domains with interruptions (Type XV, XVIII), and other types of collagen (Types VI, VII). The collagen, in one aspect, can be maintained with a triple helix structure and with epitopes without any chemical or enzyme or heat or lower pH treatments.

The phrase “effective amount” means an amount of a compound that promotes, improves, stimulates, or encourages a response to the particular condition or disorder or the particular symptom of the condition or disorder.

As used herein, the term “biomarker” refers to a measurable substance in a mammal whose presence, concentration or amount is indicative of some phenomenon such as disease, infection or inflammation and can be a joint biomarker an inflammation biomarker or the like.

As used herein, by “active” or “active ingredient” is meant a drug, medicament, pharmaceutical, therapeutic agent, nutraceutical, or other compound that may be desired to be administered to the body. The active ingredient may be a “small molecule,” generally having a molecular weight of 2000 Daltons or less. The active ingredient may also be a “biological active.” Biological active ingredients include proteins, antibodies, antibody fragments, peptides, oligonucleotides, vaccines, and various derivatives of such materials. In one embodiment, the active ingredient is a small molecule. In another embodiment, the active ingredient is a biological active. In still another embodiment, the active ingredient is a mixture of a small molecule and a biological active. Also as used herein, the terms “active ingredient”, “first active ingredient”, “second active ingredient”, etc. may be used to denote active ingredients located in different places within the particle, such as those located in the core or those located in the one or more outer layers. However, the terms “first” or “second” do not necessarily denote that the first active ingredient is different from the second active ingredient. For example, in certain embodiments, the active ingredient contained within the core may be the same as the second active ingredient contained within an outer layer disposed on the core. While in certain other embodiments, the active ingredient contained within the core may be different from the second active ingredient contained within an outer layer disposed on the core.

A molten mixture is formed by mixing and heating the lipid matrix compositions as previously described. “Molten mixture” means that the mixture of an active ingredient and lipid matrix materials are sufficiently mixed and heated to fluidize the mixture sufficiently to allow it to be atomized into droplets. Generally, the mixture is molten in the sense that it will flow when subjected to one or more forces such as pressure, shear, and centrifugal force, such as that exerted by a centrifugal or spinning-disk atomizer.

As used herein, the term “flow point” is the temperature at which any portion of the mixture becomes sufficiently fluid that the mixture, as a whole, may be atomized. Generally, a mixture is sufficiently fluid for atomization when the viscosity of the molten mixture is less than 20,000 cp, or less than 15,000 cp, or less than 10,000 cp, less than 5000 cp, or even less than 1000 cp. The viscosity can be measured by a controlled stress rheometer, which measures viscosity as a function of temperature, and may use either a shear-type or rotational rheometer. As used herein, melting point refers to the temperature that marks the midpoint of the transition from a solid crystalline or semi-crystalline state to a liquid state. As measured by DSC, the melting point is the temperature where upon heating the solid material, the maximum exothermic heat flow occurs. In general, melting point will be used in reference to relative pure single component materials such as some actives or essentially single component excipients (e.g. stearyl alcohol) and flow point will be used in reference to multi-component materials or mixtures.

As used herein, the term “semi-solid” is a solid at ambient temperature (23° C.) but becomes a liquid at temperatures above 30° C. or 40° C., or at body temperature.

Unless otherwise indicated, “capsule” means a container suitable for enclosing solids or liquids and includes empty capsule shells and components thereof such as caps and bodies that may be assembled together to form the capsule.

Unless otherwise indicated, “dosage form” refers to a solid composition comprising an active ingredient.

As used herein, the term “particle” refers a portion or quantity of material(s), such as a small portion or quantity of material(s). For example, as provided herein, the term particle may refer generally to a composition containing a core and one or more outer layers surrounding the core. In some embodiments, the particle(s) described may be generally spherical in shape. The term “particle” as used herein includes or may be used interchangeably with the following: pellet, beadlet, multiparticulates, particulates, spheres, including microspheres, seeds, and the like. The term particle as used herein is not limited to only a particle formed by certain methods or processes. Indeed, the particle(s) described herein may be formed by any suitable process. Certain suitable processes include, but are not limited to, melt spray congealing, spheronization, extrusion, compression, powder layering, liquid layering, pelletization by melt and wet granulation, and combinations thereof. The particle(s) as described herein may be solid or semi-solid particles. In some embodiments, the particles describe herein can include both solid and semi-solid compositions contained on or within the particle itself

Unless otherwise noted, lipid multiparticulates containing at least one anti-inflammatory adjuvant as discussed herein, may be placed into a capsule, formed into a tablet, placed in a softgel, placed in a gummy, may be alternatively ingested directly by a mammal as a powder or can be incorporated into a beverage or other food item. The lipid multiparticulate particles include a lipid matrix that, in one embodiment, can be formulated to release the anti-inflammatory adjuvant when the particles are in contact within an environment which cause the anti-inflammatory adjuvant to be released from the lipid multiparticulates, such as in the digestive systems of a mammal that has been orally administered or otherwise ingested the lipid multiparticulates.

As used herein, percent change in any one or more of the discussed biomarkers is based upon a baseline value for the respective subject and/or marker and does not refer to a percent change from a midpoint or non-starting point. Therefore, as used herein, “baseline” refers to a day zero or starting point, and can therefore be used to determine a value of a measured marker at a starting point, which is then used to calculate the percent change in any of the biomarkers discussed herein from the baseline.

Other features and aspects of the present disclosure are discussed in greater detail below.

### DETAILED DESCRIPTION

It is to be understood by one of ordinary skill in the art that the present discussion is a description of exemplary embodiments only and is not intended as limiting the broader aspects of the present disclosure.

Generally speaking, the present disclosure has surprisingly found that a joint health composition that includes a combination of collagen, such as undenatured type II collagen in one aspect, and an anti-inflammatory adjuvant provides one or more of reduced pro-cytokines, reduced pain, and reduced inflammation, including subjective reduction in inflammation and/or joint pain, or combinations thereof, even at low dosages and administration time periods where the anti-inflammatory adjuvant is present in a lipid multiparticulate. Namely, as will be discussed in greater detail below, the present disclosure has found that certain anti-inflammatory adjuvants in a lipid multiparticulate dosage form in combination with collagen, allows increased bioavailability and nutrient efficacy without large dosages or extensive administration times.

The joint health composition of the present disclosure, when administered to mammals suffering from joint pain and/or inflammation, can reduce joint pain and inflammation in skeletal joints. When administered to mammals suffering from joint pain and/or inflammation, the joint health composition can dramatically decrease serum interleukin-1 levels in a mammal suffering from joint pain and/or inflammation. Serum interleukin levels, such as interleukin-1, can decrease by greater than about 10%, such as greater than about 20%, such as greater than about 30%, such as greater than about 40%, and generally less than about 100%. The above reductions can occur after a period of time of being administered the joint health composition at least once every three days, such as at least every day. Moreover, as discussed above, the above results also occur more quickly than previous compositions and are therefore considered to be “fast acting”. Thus, in one aspect, the dramatic reduction in inflammation and joint pain, for instance, can also be exhibited even after only two weeks or less of administration, such as after only 10 days or less of administration, such as after only 1 week or less of administration, such as after only 5 days or less of administration, such as after as little as 3 days or less of administration. However, it should be understood that the joint health composition of the present disclosure also provides long-term benefits, as the above, or any of the following benefits are also exhibited over the course of supplementation with the joint health composition. Thus, in one aspect, the reduction in inflammation and joint pain for any of the biomarkers discussed herein are exhibited for about 2 weeks or more, such as about 1 month or more, such as about 6 weeks or more, or for any of the supplementation times discussed herein.

The joint health composition of the present disclosure, when administered to mammals suffering from joint pain and/or inflammation, can reduce joint pain and inflammation in skeletal joints. When administered to mammals suffering from joint pain and/or inflammation, the joint health composition can dramatically decrease serum interleukin-6 levels in a mammal suffering from joint pain and/or inflammation. Serum interleukin levels can decrease by greater than about 10%, such as greater than about 20%, such as greater than about 30%, such as greater than about 40%, and generally less than about 120%. The above reductions can occur after a period of time of being administered the joint health composition at least once every three days, such as at least every day. Moreover, as discussed above, the above results also occur more quickly than previously compositions and are therefore considered to be “fast acting”. Thus, in one aspect, the dramatic reduction in inflammation and joint pain, for instance, can also be exhibited even after only two weeks or less of administration, such as after only 10 days or less of administration, such as after only 1 week or less of administration, such as after only 5 days or less of administration, such as after as little as 3 days or less of administration.

The joint-health composition of the present disclosure, when administered to mammals suffering from joint pain and/or inflammation, can reduce joint pain and inflammation in skeletal joints. When administered to mammals suffering from joint pain and/or inflammation, the joint health composition can dramatically reduce tumor necrosis factor alpha (TNF α), which is believed to play an important role in inflammation by initiating an inflammatory cascade. The method of the present disclosure, however, can reduce serum TNF α in the body of the mammal by greater than about 5%, such as greater than about 15%, such as greater than about 25%, and generally less than about 120%, such as less than about 80%. Similarly, TNF α protein can be reduced within the body of the mammal by greater than about 10%, such as by greater than about 20%, such as greater than about 25%, such as greater than about 35%, and generally less than about 80%. The above reductions can occur after a period of time of being administered the joint health composition at least once every three days, such as at least every day. Moreover, as discussed above, the above results also occur more quickly than previously compositions and are therefore considered to be “fast acting”. Thus, in one aspect, the dramatic reduction in inflammation and joint pain, for instance, can also be exhibited even after only two weeks or less of administration, such as after only 10 days or less of administration, such as after only 1 week or less of administration, such as after only 5 days or less of administration, such as after as little as 3 days or less of administration.

The joint-health composition of the present disclosure, when administered to mammals suffering from joint pain and/or inflammation, can reduce joint pain and inflammation in skeletal joints. When administered to mammals suffering from joint pain and/or inflammation, the joint health composition can dramatically decrease serum cyclooxygenase-2 (COX-2) protein levels in a mammal suffering from joint pain and/or inflammation. COX-2 levels can decrease by greater than about 10%, such as greater than about 20%, such as greater than about 30%, such as greater than about 40%, and generally less than about 50%. %. The above reductions can occur after a period of time of being administered the joint health composition at least once every three days, such as at least every day. Moreover, as discussed above, the above results also occur more quickly than previously compositions and are therefore considered to be “fast acting”. Thus, in one aspect, the dramatic reduction in inflammation and joint pain, for instance, can also be exhibited even after only two weeks or less of administration, such as after only 10 days or less of administration, such as after only 1 week or less of administration, such as after only 5 days or less of administration, such as after as little as 3 days or less of administration.

The joint-health composition of the present disclosure, when administered to mammals suffering from joint pain and/or inflammation, can reduce joint pain and inflammation in skeletal joints. When administered to mammals suffering from joint pain and/or inflammation, the joint health composition can dramatically decrease SRY-Box Transcription Factor 9 (SOX-9) levels in a mammal suffering from joint pain and/or inflammation. SOX-9 levels can decrease by greater than about 8%, such as greater than about 15%, such as greater than about 25%, such as greater than about 30%, and generally less than about 50%. The above reductions can occur after a period of time of being administered the joint health composition at least once every three days, such as at least every day. Moreover, as discussed above, the above results also occur more quickly than previously compositions and are therefore considered to be “fast acting”. Thus, in one aspect, the dramatic reduction in inflammation and joint pain, for instance, can also be exhibited even after only two weeks or less of administration, such as after only 10 days or less of administration, such as after only 1 week or less of administration, such as after only 5 days or less of administration, such as after as little as 3 days or less of administration.

The joint-health composition of the present disclosure, when administered to mammals suffering from joint pain and/or inflammation, can reduce joint pain and inflammation in skeletal joints. When administered to mammals suffering from joint pain and/or inflammation, the joint health composition can dramatically decrease Matrix Metallopeptidase 13 (MMP-13) levels in a mammal suffering from joint pain and/or inflammation. MMP-13 protein levels can decrease by greater than about 8%, such as greater than about 10%, such as greater than about 15%, such as greater than about 20%, and generally less than about 60%. The above reductions can occur after a period of time of being administered the joint health composition at least once every three days, such as at least every day. Moreover, as discussed above, the above results also occur more quickly than previously compositions and are therefore considered to be “fast acting”. Thus, in one aspect, the dramatic reduction in inflammation and joint pain, for instance, can also be exhibited even after only two weeks or less of administration, such as after only 10 days or less of administration, such as after only 1 week or less of administration, such as after only 5 days or less of administration, such as after as little as 3 days or less of administration.

As shown above, the effect on biomarkers within the mammal can be dramatic and far exceed biomarker influence recorded in the past when a mammal has been administered collagen or an anti-inflammatory adjuvant alone, or without the anti-inflammatory adjuvant being in the delivery form of a lipid multiparticulate. Furthermore, as discussed above, the joint-health composition of the present disclosure exhibits an improvement in joint health or reduced inflammation as evidenced by one or more of the above factors more quickly and/or effectively than previous supplements. Thus, as discussed above, the joint-health composition of the present disclosure can be considered to be “fast acting” and exhibit improvements in as little as two weeks of administration, such as after only 10 days or less of administration, such as after only 1 week or less of administration, such as after only 5 days or less of administration, such as after as little as 3 days or less of administration. Furthermore, as may be discussed in greater detail below, it should be understood based upon the discussion of the benefits of the joint health composition of the present disclosure, that bioavailability of the components of the joint health composition is unexpectedly improved, including where the joint health composition is tailored for delayed release, sustained release, or steady release formulations.

However, as noted above, in one aspect, it should be understood that the reduction joint pain and/or inflammation is a subjective perception by the mammal of an improvement in joint pain and/or inflammation, and does not require measurement of one or more inflammatory markers.

As described above, the joint health composition of the present disclosure contains collagen in combination with an anti-inflammatory adjuvant, and well as any other optional various additives and ingredients. The joint health composition can include one or more of any collagen as defined above, and/or, in one aspect, may include one or more of Type I collagen, Type II collagen, Type III collagen, Type IV collagen, or collagen peptides, or a mixture thereof. In one aspect, the joint health composition contains Type II collagen alone or in combination with one or more of Type I collagen, Type III collagen, Type IV collagen, or collagen peptides. In one aspect, the joint health composition may include a mixture of Type II collagen (sometimes referred to as native Type II collagen) and undenatured Type II collagen. Additionally or alternatively, the joint health composition may include a mixture of native Type II collagen and undenatured Type II collagen, in addition to a further collagen, such as Type I, Type III, Type IV, or collagen peptides. Furthermore, in one aspect, the joint health composition includes whole collagen protein, biologically active peptide fragments of collagen, or a combination thereof.

In one aspect, one or more types of collagen in the joint health composition may be heat sterilized, such as by autoclaving, and/or may also include salts, such as alkalizing or acid, and/or organic or inorganic salts. Thus, in one aspect, a portion of the collagen may be at least partially hydrolyzed. In one aspect, the collagen is hydrolyzed by any process or compound, including by an acid base agent, an enzyme, heat or other temperature extreme, a chemical, UV, a salt, or combinations thereof. In one aspect, the hydrolyzed collagen is undenatured collagen that has been at least partially hydrolyzed by any method, and in one aspect, may include undenatured collagen that has any portion that has been denatured. However, as discussed, in one aspect, at least a portion of the joint health composition is undenatured.

As indicated above, in one aspect, the joint-health composition contains an undenatured collagen, particularly a Type II collagen such as an undenatured Type II collagen, an at least partially hydrolyzed Type II collagen, or a combination thereof. Type II collagen for use in the present disclosure can be obtained from any suitable source. For instance, the collagen can be derived from a variety of mammalian sources, avian sources, or can be obtained from various fish species or a combination thereof. For instance, the collagen can be obtained from sea and/or animal sources, such as salmon, shark, poultry, porcine, eggshells, turkey cartilage, bovine cartilage, and the like. In one embodiment, for instance, the Type II collagen can be obtained as disclosed in U.S. Pat. No. 7,083,820 to Schilling which is incorporated by reference. For example, undenatured Type II collagen is available commercially as UC-II® brand from Lonza Consumer Health Inc. UC-II® brand is a natural ingredient that contains a glycosylated, undenatured Type II collagen. The joint health composition can also comprise a hydrolyzed collagen. The joint health composition can also comprise a pure protein or active peptide fragments. In one embodiment, the joint health composition can be free of any bone or bone material. In other embodiments, the joint health composition can be free of any transforming growth factors (TGFs), bone morphogenetic proteins (BMPs), or both. In still another embodiment, the joint health composition comprises Type II collagen and is completely free of any Type I collagen.

In preparing animal tissue for oral administration, in one embodiment, the Type II collagen containing tissue can be first dissected free of surrounding tissues and diced or otherwise comminuted into particles. The particulate, or milled, cartilage can be sterilized by means which do not affect or denature the structure of a major portion of the type II collagen in the tissue, such as low-temperature processing, and formed into doses containing therapeutically effective levels of undenatured type II collagen. Being a natural product some variation from sample to sample is to be expected. These variations can be minimized by blending after comminution. The blending can be aided by analytical techniques which allow the measurement of the amount of undenatured type II collagen and other constituents.

Nonetheless, the present disclosure has found that by carefully forming the type II collagen as discussed above, the undenatured type II collagen may be resistant to gastric acid and digestive enzymes in the stomach. Due to this sterilization process, the undenatured type II collagen also retains its 3-dimensional shape, preserving the bioactive epitope regions. Without wishing to be bound by theory, it is believed that the epitope regions contain the ability to induce oral tolerance as discussed above. Particularly epitope regions allow undenatured collagen to bind to the Peyer's Patches, which have the ability to induce oral tolerance processes.

The amount of collagen contained in the joint-health composition administered to a mammal, the number of doses per unit time, and the length of the treatment can all vary depending upon various factors including the type of mammal being treated, the injury or ailment that is being treated, and the like. In one embodiment, for instance, the joint health composition is administered to the mammal such that the mammal receives collagen in an amount of at least 0.3 mg/kg of body weight per day, such as an amount greater than about 0.35 mg/kg of body weight per day, such as greater than about 0.4 mg/kg of body weight per day, such as greater than about 0.45 mg/kg of body weight per day, such as greater than about 0.5 mg/kg of body weight per day, such as greater than about 0.65 mg/kg of body weight per day, such as greater than about 0.75 mg/kg of body weight per day, such as greater than about 1 mg/kg of body weight per day, such as greater than about 1.5 mg/kg of body weight per day, such as greater than about 2 mg/kg of body weight per day, such as greater than about 2.2 mg/kg of body weight per day, and generally less than about 5 mg/kg of body weight per day, such as less than about 4 mg/kg of body weight per day.

In one particular embodiment, collagen is administered to the mammal in an amount of from about 0.3 mg/kg of body weight per day to about 3 mg/kg of body weight per day, such as about 0.4 mg/kg of body weight per day to about 2.5 mg/kg of body weight per day, such as about 0.5 mg/kg of body weight per day to about 2 mg/kg of body weight per day, or any ranges or values therebetween.

The joint-health composition of the present disclosure contains collagen, such as any suitable collagen in various forms. In one embodiment, the collagen contained in the joint health composition is an undenatured Type II collagen. Collagen has been used in the past as a supplement to improve joint health in healthy mammals. For example, PCT Application No. PCT/US2021/045853, which was filed on Aug. 13, 2021, and which is incorporated herein by reference, discloses a supplement and method for improving inflammation, joint health, joint mobility, and joint comfort in healthy mammals. In the past, it was believed that the benefits of administering collagen to a mammal were realized at lower dose amounts and that increasing the amount of collagen administered to the mammal had no additional benefits. To the contrary, it has now been discovered that administering greater amounts of collagen to a mammal can also provide benefits to a mammal, such as to improve skeletal articulation health in mammals suffering from ailments and diseases such as osteoarthritis and/or skeletal necrosis.

For example, in the past, it was widely believed that daily collagen doses of greater than about 0.55, such as greater than about 0.6 mg/kg of body weight per day (e.g. 40 mg of collagen for some mammals such as humans) would have no added beneficial effect on the user or mammal. It was surprisingly and unexpectedly discovered, however, that administering greater than about 0.66 mg/kg of body weight per day of collagen can have beneficial effects on joint injuries or diseases. However, as discussed above, it should also be understood that, when used in combination with the anti-inflammatory adjuvant, less than 0.6 mg/kg of body weight per day of collagen can also provide the beneficial effects noted herein, thus, as noted above, in one aspect, the method of the present disclosure includes administering less than 0.3 mg/kg of body weight per day of the joint health composition, such as less than 0.5 mg/kg of body weight per day of the joint health composition, such as less than 0.6 mg/kg of body weight per day of the joint health composition.

When administering collagen in accordance with the present disclosure to larger mammals including humans, collagen can be contained in the joint health composition sufficient to be administered to the mammal in an amount greater than about 50 mg per day, such as in an amount greater than about 80 mg per day, such as in an amount greater than about 120 mg per day, such as in an amount greater than about 160 mg per day, such as in an amount greater than about 320 mg per day, such as in an amount greater than about 480 mg per day, such as in an amount greater than about 640 mg per day, such as in an amount greater than about 800 mg per day. The amount of collagen administered to the mammal is generally less than about 5,000 mg per day, such as less than about 3,000 mg per day, such as less than about 2,000 mg per day, such as less than about 1,000 mg per day, such as less than about 500 mg per day. It should also be understood that the above values can also include an amount administer to a mammal.

The joint health composition can be administered to the mammal orally and can be in the form of a nutraceutical supplement in the dosage form of a lipid multiparticulate (LMP) as will be discussed in greater detail below. The supplement can be in the form of individual dosage vessels that can be swallowed, chewed, drank, or the like. The amount of collagen in each dosage vessel, in one aspect can be in the same amounts discussed in regards to daily dosage above, or can be from about 10 mg to about 1000 mg, such as about 20 mg to about 900 mg, such as about 40 mg to about 800 mg, such as about 50 mg to about 640 mg, such as about 120 to about 320 mg, or any ranges or values therebetween. In one aspect, each dosage vessel as described above can be administered to the mammal once daily, or at least twice daily, such as at least three times daily, such as at least four times daily, and generally less than about eight times daily, such as less than about six times daily, such as less than about five times daily.

In one aspect, undenatured Type II collagen may form all, or substantially all, of the total Type II collagen of the above discussed collagen, and therefore, may be present in the supplement in the above discussed amounts. However, in one aspect, undenatured Type II collagen may account for about 0.5% to about 95% of the total Type II collagen and/or collagen composition, such as about 1% to about 75%, such as about 1.5% to about 50%, such as about 2% to about 40%, such as about 2.5% to about 15% of the total Type II collagen or total collagen composition, or any ranges or values therebetween. Therefore, in one aspect, undenatured type II collagen may be present in the composition in an amount of 0.1 mg to about 100 mg, such as about 0.5 mg to about 75 mg, such as about 0.75 mg to about 50 mg, such as about 1 mg to about 30 mg, or any ranges or values therebetween.

In one aspect, the collagen may further include a preservative salt or sugar, or other food preservative agents, such as potassium chloride for example. Thus, in one aspect, the total amounts of collagen discussed above may include Type II collagen and/or undenatured Type II collagen, alone or in combination with a further collagen, a preservative salt, or combinations thereof. In such as aspect, the total Type II collagen, including native and undenatured Type II collagen, may account for about 1% to about 99% of the collagen composition, such as about 2.5% to about 90%, such as about 5% to about 80%, such as about 7.5% to about 70%, such as about 10% to about 60%, such as about 15% to about 50%, such as about 20% to about 35%, or any ranges or values therebetween.

When the Type II collagen includes undenatured Type II collagen, the undenatured Type II collagen may have a large oxygen radical absorbance capacity (ORAC), as measured according to ORAC 6.0. Particularly, ORAC tests measure antioxidant scavenging activity against oxygen radicals that are known to be involved in the pathogenesis of aging and common disease, and consist of six types of ORAC assays that evaluate the antioxidant capacity of a material against primary reactive oxygen species, peroxyl radical, hydroxyl radical, superoxide anion, and peroxynitrite. Particularly, the ORAC assay includes introducing a reactive oxygen species (ROS) introducer to the assay system, where the ROS introducer triggers the release of a specific ROS which would degrade the probe and cause its emission wavelength or intensity to change. Thus, if the assay being tested includes an antioxidant, the antioxidant absorbs the ROS and preserves the probe from degradation. The degree of probe preservation indicates the antioxidant capacity of the material, and the results are expressed as μmol trolox equivalents (TE)/g of a tested material.

For example, an ORAC assay against peroxyl radical measures the antioxidant capacity of a sample to protect the fluorescent protein (fluorescein) from damage by a peroxyl radical which is generated from 2,2′ azobis(2 amidinopropane) dihydrochloride (AAPH). The ORAC assay against hydroxyl radical measures the antioxidant capacity of the sample to protect the fluorescent protein (fluorescein) from damage by a hydroxyl radical which is generated from reaction between cobalt and hydrogen peroxide. The ORAC assay against peroxynitrite measures the antioxidant capacity of the sample to protect Dihydrorhodamine-123 from damage by a peroxynitrite radical which is generated from 3-morpholinosyndnonimine hydrochloride. The ORAC assay against superoxide measures the antioxidant capacity of the sample to protect hydroethidine from damage by a superoxide which is generated from xanthine oxidase. The ORAC assay against singlet oxygen measures the antioxidant capacity of the sample to protect hydroethidine from damage by single oxygen which is generated from a reaction between lithium molybdate and hydrogen peroxide. Finally, the ORAC assay against hypochlorite measures the antioxidant capacity of the sample to protect the fluorescent protein fluorescein from damage by the hypochlorite radical which is generated from sodium hypochlorite.

Thus, in one aspect, a collagen composition having an undenatured Type II collagen according to the present disclosure may have a total ORAC of about 200 μmol TE/g or greater, such as about 250 μmol TE/g or greater, such as about 300 μmol TE/g or greater, such as about 350 μmol TE/g or greater, such as about 400 μmol TE/g or greater, such as about 450 μmol TE/g or greater, such as about 500 μmol TE/g or greater, such as about 550 μmol TE/g or greater, such as about 600 μmol TE/g or greater, such as about 700 μmol TE/g or greater, such as about 750 μmol TE/g or greater, such as about 800 μmol TE/g or greater, such as about 825 μmol TE/g or greater, up to about 1000 μmol TE/g, or any ranges or values therebetween.

Furthermore, in one aspect, a collagen composition having an undenatured Type II collagen according to the present disclosure may have a ORAC against peroxyl radicals of about 1 μmol TE/g or greater, such as about 2.5 μmol TE/g or greater, such as about 5 μmol TE/g or greater, such as about 7.5 μmol TE/g or greater, such as about 10 μmol TE/g or greater, such as up to about 10.5 μmol TE/g or greater, up to about 50 μmol TE/g, or any ranges or values therebetween.

Similarly, in one aspect, a collagen composition having an undenatured Type II collagen according to the present disclosure may have a ORAC against hydroxyl radicals of about 10 μmol TE/g or greater, such as about 15 μmol TE/g or greater, such as about 20 μmol TE/g or greater, such as about 25 μmol TE/g or greater, such as about 27.5 μmol TE/g or greater, such as about 30 μmol TE/g or greater, up to about 40 μmol TE/g, or any ranges or values therebetween.

Additionally or alternatively, in one aspect, a collagen composition having an undenatured Type II collagen according to the present disclosure may have a ORAC against peroxynitrite of about 0.5 μmol TE/g or greater, such as about 1 μmol TE/g or greater, such as about 1.5 μmol TE/g or greater, such as about 2 μmol TE/g or greater, such as about 2.25 μmol TE/g or greater, up to about 5 μmol TE/g, or any ranges or values therebetween.

In one aspect, a collagen composition having an undenatured Type II collagen according to the present disclosure may have a ORAC against singlet oxygen of about 500 μmol TE/g or greater, such as about 550 μmol TE/g or greater, such as about 600 μmol TE/g or greater, such as about 650 μmol TE/g or greater, such as about 700 μmol TE/g or greater, such as about 725 μmol TE/g or greater, up to about 1000 μmol TE/g, or any ranges or values therebetween.

Furthermore, in one aspect, a collagen composition having an undenatured Type II collagen according to the present disclosure may have a ORAC against hypochlorite of about 25 μmol TE/g or greater, such as about 30 μmol TE/g or greater, such as about 35 μmol TE/g or greater, such as about 40 μmol TE/g or greater, such as about 45 μmol TE/g or greater, such as up to about 50 μmol TE/g or greater, up to about 75 μmol TE/g, or any ranges or values therebetween.

Furthermore, in one aspect, when the Type II collagen includes undenatured Type II collagen, the undenatured Type II collagen may have a molecular weight of about 10,000 Daltons or more, such as about 15,000 Daltons or more, such as about 20,000 Daltons or more, such as about 25,000 Daltons or more, such as about 30,000 Daltons or more, such as about 35,000 Daltons or more, such as about 40,000 Daltons or more, such as about 45,000 Daltons or more, such as about 50,000 Daltons or more, such as about 55,000 Daltons or more, such as about 60,000 Daltons or more, such as about 65,000 Daltons or more, such as about 70,000 Daltons or more, such as about 75,000 Daltons or more, such as about 80,000 Daltons or more, such as about 85,000 Daltons or more, such as about 90,000 Daltons or more such as about 95,000 Daltons or more, such as about 100,000 Daltons or more, up to about 350,000 Daltons or less, or any ranges or values therebetween.

Nonetheless, as discussed above, the joint health composition of the present disclosure also includes at least one anti-inflammatory adjuvant which can act synergistically with the collagen to improve joint pain and inflammation, as well as decrease the amount of time needed to improve the joint pain and inflammation. In one aspect, the joint-health adjuvant can be an extract and/or isolate from a natural source, such as a plant or animal, that exhibits joint health or inflammation reducing properties, such as an extract or isolate from a natural source that inhibits one or more of: interleukin, including interleukin-1 and/or interleukin-6, NF-KB, cyclooxygenase-2 (COX-2) expressions, inducible nitric oxide synthase (iNOS), 5-lipoxygenase biosynthesis, and tumor necrosis factor-α. Thus, in one aspect, the plant or animal from which the extract and/or isolate is obtained is Andrographis paniculate, Tamarind seed, Boswellia serrata, green lipped mussel (Perna canaliculus), turmeric (Curcuma longa), stinging nettle (Urtica dioica), Cat's claw (Uncaria tomentosa), bromelain, willow bark, or combinations thereof. Nonetheless, as will be discussed in greater detail below, in one aspect, the anti-inflammatory adjuvant is a boswellic acid, including 11-keto-β-boswellic acid and/or acetyl-11-keto-β-boswellic acid, tamarind seed extract (TSE), andrographolide, or combinations or derivatives thereof.

Furthermore, in one aspect, the joint health composition can include an isolate and/or extract from Andrographis paniculate, Tamarind seed, green lipped mussel (Perna canaliculus), turmeric (Curcuma longa), stinging nettle (Urtica dioica), Cat's claw (Uncaria tomentosa), and bromelain, as defined above, or in addition to/outside of the LMP, in combination with a collagen, which can be undenatured collagen, as discussed herein.

Regardless of the anti-inflammatory adjuvant selected, in one aspect, the anti-inflammatory adjuvant is present in the joint-health composition in an amount of about200 mg or less, such as about 175 mg or less, such as about 150 mg or less, such as bout 125 mg or less, 100 mg or less, such as about 90 mg or less, such as about 80 mg or less, such as about 70 mg or less, such as about 60 mg or less, such as about 50 mg or less, such as about 40 mg or less, such as about 35 mg or less, such as about 30 mg or less, such as about 25 mg or less, such as about 20 mg or less, such as about 15 mg or less, such as about 10 mg or less, such as about 2.5 mg or more, or any ranges or values therebetween. Stated differently, the anti-inflammatory adjuvant can be present in the joint-health composition such that a mammal, which in one aspect may be a human, receives about 1.66 mg/kg of body weight per day or less, such as about 1.5 mg/kg or less, such as about 1.25 mg/kg or less, such as about 1 mg/kg or less, such as about 0.75 mg/kg or less, such as about 0.5 mg/kg of body weight or less, such as about 0.1 mg/kg of body weight or greater, or any ranges or values therebetween.

In one aspect, when TSE is used alone or in combination with one or more anti-inflammatory adjuvants, the TSE is present in the joint-health composition in an amount of about 10 mg or more, such as about 25 mg or more, such as about 30 mg or more, such as about 40 mg or more, such as about 50 mg or more, such as about 60 mg or more, up to about 100 mg or less, or any ranges or values therebetween. Stated differently, the TSE can be present in the joint-health composition such that a mammal receives about 0.15 mg/kg of body weight per day or more, such as about 0.4 mg/kg or more, such as about 0.66 mg/kg or more, such as about 0.8 mg/kg or more, such as about 0.75 mg/kg or more, such as about 1 mg/kg of body weight or more, such as about 1.66 mg/kg of body weight or less, or any ranges or values therebetween.

Furthermore, when andrographolide is used alone or in combination, the andrographolide is present in the joint-health composition in an amount of about 2.5 mg or more, such as about 5 mg or more, such as about 7.5 mg or more, such as about 10 mg or more, such as about 12.5 mg or more, such as about 15 mg or more, such as about 17.5 mg or more, such as about 20 mg or more, such as about 22.5 mg or more, up to about 25 mg or less, or any ranges or values therebetween. Stated differently, the andrographolide can be present in the joint-health composition such that a mammal receives about 0.05 mg/kg of body weight per day or more, such as about 0.1 mg/kg or more, such as about 0.15 mg/kg or more, such as about 0.2 mg/kg or more, such as about 0.25 mg/kg or more, such as about 0.3 mg/kg of body weight or more, such as about 0.4 mg/kg of body weight or less, or any ranges or values therebetween.

Moreover, in one aspect where boswellic acid is used alone or in combination, the boswellic acid is present in the joint-health composition in an amount of about 5 mg or more, such as about 10 mg or more, such as about 15 mg or more, such as about 20 mg or more, such as about 25 mg or more, such as about 30 mg or more, such as about 35 mg or more, such as about 40 mg or more, up to about 45 mg or less, or any ranges or values therebetween. Stated differently, the boswellic acid can be present in the joint-health composition such that a mammal receives about 0.05 mg/kg of body weight per day or more, such as about 0.1 mg/kg or more, such as about 0.2 mg/kg or more, such as about 0.3 mg/kg or more, such as about 0.4 mg/kg or more, such as about 0.5 mg/kg of body weight or more, such as about 0.75 mg/kg of body weight or less, or any ranges or values therebetween.

Regardless of the anti-inflammatory adjuvant and collagen selected, in one aspect, the joint health composition includes a total amount of collagen and anti-inflammatory adjuvant of about 400 mg or less, such as about 350 mg or less, such as about 300 mg or less, such as about 250 mg or less, such as about 225 mg or less, such as about 210 mg or less, such as about 200 mg or less, or any ranges or values therebetween. Furthermore, in one aspect, as discussed above, the joint-health composition is suitable for administration once per day or more. Thus, in such an aspect, the above amounts are the total amounts per day administered to a mammal, or may be multiplied based upon the number of dosages supplied per day. However, in one aspect, it should be understood that only one dosage may be applied per day to achieve the effects discussed herein. Namely, as discussed above, the present disclosure has surprisingly found that the combination of the collagen and the anti-inflammatory adjuvant when applied in a LMP delivery form can exhibit the above effects without increasing the dosage, and instead, exhibits improvements in joint health and inflammation at small daily dosages. Furthermore, it should be understood that additional ingredients, as discussed below in greater detail, may be contained in the joint health composition, optionally within the lipid multiparticulate, in amounts according to the above, or greater (e.g. up to about 1 gram or greater, or according to the above amounts).

Thus, as discussed above, in one aspect, the joint-health composition contains the anti-inflammatory adjuvant in the form of a lipid multiparticulate (LMP), for instance, dispersed in the lipid matrix of the LMP. Namely, as will be discussed in greater detail in the examples below, the present disclosure has found that the bioavailability and bioactive nutrient efficacy of the joint-heath composition is dramatically increased by incorporation of the anti-inflammatory adjuvant in the form of a LMP, further improving the efficacy per dosage size. For instance, such an aspect may include one or more particles, wherein each of the particles includes a core containing a lipid matrix having the composition dispersed therein, as the composition may be a fat-soluble active ingredient. The plurality of particles may also include one or more outer layers disposed thereon that may include one or more active ingredients, which may include the anti-inflammatory adjuvant, or which may be a further active ingredient. The plurality of particles may be incorporated into a capsule or any other suitable oral or other dosage form discussed in greater detail below.

In one aspect utilizing a LMP delivery, the lipid matrix of the core may be formulated such that the core contains from about 10% to about 60% by volume of the composition. For example, in certain embodiments, the core may contain at least about 15% by volume of the composition, such as at least about 20% by volume, such as at least about 25% by volume, such as at least about 30% by volume, such as at least about 35% by volume, such as at least about 40% by volume, such as at least about 45% by volume, such as at least about 50% by volume, or any ranges or values therebetween.

In certain aspects, the lipid matrix of the core may include a) at least one low flow point excipient, b) at least one high flow point excipient, c) at least one low-flow point surfactant, and c) optionally an antioxidant.

The cores disclosed herein may include a lipid matrix that contains a low-flow point excipient. For example, in certain embodiments the lipid matrix may contain one or more low-flow point excipients. Low flow point excipients generally include fatty alcohols, fatty acids, fatty acid esters of glycols and poly glycols, fatty acid esters of polyglycerol and fatty acid esters of glycerol (glycerides) with flow points of less than 50° C. When the low flow point excipient is a relatively pure material, the melting point is also less than 50° C. A preferred class of low flow point excipients are low flow point glycerides. By “low flow point” excipient, such as a glyceride, is meant that the melting point of the excipient, such as a glyceride, is less than 50° C. In some embodiments, the low flow point glyceride has a melting point of less than 40° C. In some embodiments, the low-flow point excipient, such as glyceride, is a mixture of compounds, having a flow point of 50° C. or less. In some embodiments, the low-flow point excipient, such as glyceride, has a flow point of 40° C. or less. In some embodiments, the low-flow point glyceride has a low flow point of 30° C. or less. Exemplary low flow point glycerides include polyglycolized glycerides, such as some of the Gelucire products manufactured by Gattefosse, such as Gelucire® 43/01 having a nominal melting point of 43° C. Mixtures of low flow point glycerides are also effective, such as mixtures of Gelucire® 43/01 (C10-C18 triglycerides), Gelucire® 50/13 (stearoyl polyoxylglycerides), Gelucire® 44/14 (lauroyl macrogol-32 glycerides), and mixtures thereof. Other glycerides may also be used, such as fatty acid esters of glycols and poly glycols, and fatty acid esters of polyglycerols.

A function of the low flow point excipient is to ensure that at least a significant portion of the formulation matrix softens when ingested orally by a patient in need of therapy, at the temperature of the Gl tract (about 37 for humans). This allows the formulation to break down by digestion in the gastro-intestinal (Gl) tract, and ultimately to disperse in the Gl tract to promote dissolution and absorption of the active. In certain embodiments the low flow point excipient provides a significant portion of the formulation matrix to be present in a non-crystalline liquid or amorphous state when ingested and softened in the Gl tract.

Exemplary low flow point fatty alcohols include myristyl alcohol (Tm 38° C.), lauryl alcohol (Tm 23° C.) and capric alcohol (Tm 7° C.). Exemplary low flow point fatty acids include lauric acid (Tm 44° C.) and oleic acid (Tm 16° C.).

In certain aspects, the cores may include a lipid matrix including a high-flow point excipient. For example, in certain embodiments the lipid matrix may contain one or more high-flow point excipients. By “high flow point” excipient is meant an excipient that has a flow point 50° C. or more. High flow point excipients may also have a melting point above 50° C. High flow point excipients generally include fatty alcohols, fatty acids, fatty acid esters of glycols and poly glycols, fatty acid esters of polyglycerol, fatty acid esters of glycerol (glycerides), waxes, including xyloglucans such as from tamarind seed, polar waxes and other materials with flow points of greater than 50. A preferred class of high flow point excipients are “high flow point glycerides”. By high flow point glyceride is meant that the flow point or melting point of the glyceride is 50° C. or more. In some embodiments, the high flow point glyceride has a melting point of 60° C. or more. In some embodiments, the high-melting point glyceride is a mixture of compounds, having a flow point of 50° C. or more. In some aspects, the high-flow point glyceride has a flow point of 60° C. or more. In some aspects, the high flow point glyceride has a flow point of 70° C. or more.

Exemplary high flow point glycerides include glycerol behenate, glycerol dibehenate, glycerol palmitate, hydrogenated castor oil, and mixtures thereof. Often, the high flow point glyceride is a mixture of compounds that are formulated into a product and sold under a variety of trade names. Furthermore, exemplary high flow point and high melt point fatty alcohols include stearyl alcohol (Tm 58° C.) and behenyl alcohol (Tm 71° C.). Exemplary high flow point and high melt point fatty acids include palmitic acid (Tm 63° C.) and stearic acid (Tm>70° C.) Additionally or alternatively, exemplary waxes include paraffin wax, beeswax, candelilla wax, carnauba wax, and mixtures thereof.

A function of the high flow point excipient is to aid in the manufacturability of the cores by enabling the cores to congeal at a lower temperature to obtain solid particles during the melt-spray-congeal processing. In certain aspects the high flow point excipient aids the physical stability of the core formulation. In most embodiments, the high flow point excipient is not appreciably digested in the Gl tract.

In some aspects, the cores or the lipid matrix of the cores may include other excipients to improve the performance and chemical stability of the formulations. In one aspect, a dispersing agent is included in the core. Exemplary dispersing agents include lecithin, glycerin monostearate, ethylene glycol palmitostearate, aluminum oxide, polyethylene alky ethers, sorbitan esters, and mixtures thereof. In one aspect, the cores include an antioxidant to maintain chemical stability of the active agent. Exemplary antioxidants include vitamin E, tocopheryl polyethylene glycol succinate (TPGS), rosemary extract, ascorbic acid, ascorbyl palmitate, butylated hydroxyanisole (BHA), buytlated hydroxytoluene (BHT), and mixtures and combinations thereof.

Furthermore, in one aspect, a flow aid is used to improve the flow properties of the cores. Exemplary flow aids also known as glidants include calcium silicate, cab-o-sil, silicon dioxide, calcium phosphate tribasic, colloidal silicone dioxide, magnesium silicate, magnesium trisilicate, starch, talc, and other flow aids.

In one aspect, the dietary composition further contains a disintegrating agent. The disintegrating agent, for example, can be a cross-linked carboxymethyl cellulose, such as croscarmellose. Croscarmellose is a cross-linked carboxymethyl cellulose salt. In one aspect, the cross-linked carboxymethyl cellulose can be a sodium salt. In one embodiment, the cross-linked carboxymethyl cellulose can be in the form of fibers or particles. The fibers or particles can form a free-flowing powder that is typically white in color. The cross-linked carboxymethyl cellulose is hydrophilic but also insoluble. Once placed in contact with a liquid, the cross-linked carboxymethyl cellulose wicks the fluid and begins to swell. The swelling action of the cross-linked carboxymethyl cellulose causes the dietary composition to disintegrate. In this manner, the cross-linked carboxymethyl cellulose can be used to control the release of the Boswellia derivative compound.

The ability of the disintegrating agent to affect release of the anti-inflammatory adjuvant can be controlled by controlling the type of cross-linked carboxymethyl cellulose incorporated into the composition and by controlling the amount of the disintegrating agent added to the composition. For example, the ability of the cross-linked carboxymethyl cellulose to swell can depend upon the hydration of the carboxymethyl groups by controlling the degree of substitution within the cross-linked cellulose polymer. The degree of substitution, for instance, can be greater than about 0.5, such as greater than about 0.55, such as greater than about 0.6, such as greater than about 0.65, such as greater than about 0.7, such as greater than about 0.75, such as greater than about 0.8. The degree of substitution is generally less than about 0.9, such as less than about 0.85, such as less than about 0.8, such as less than about 0.75. The degree of substitution can be determined by elemental analysis.

The amount of the disintegrating agent or the cross-linked carboxymethyl cellulose incorporated into the dietary composition can generally be greater than about 0.5% by weight, such as greater than about 1% by weight, such as greater than about 3% by weight, such as greater than about 5% by weight, And generally less than about 15% by weight, such as less than about 12% by weight, such as less than about 10% by weight, such as less than about 8% by weight.

The cores described herein are generally a plurality of particles or beadlets that are solid at ambient temperature and are generally spherical in shape. By generally spherical is meant that while most particles are essentially spherical, they do not necessarily form “perfect” spheres. Such particle variations in spherical shapes are known to those persons of ordinary skill in the art of melt-spray-congeal processing and similar particulate forming methods.

The cores may have a size ranging from a mean diameter of about 40 μm to about 3000 μm, such as from about 50 μm to about 2500 μm, such as from about 80 μm to about 2000 μm, such as from about 100 μm to about 1500 μm, such as from about 200 μm to about 1000 μm, such as from about 300 μm to about 800 μm. To measure the diameters of the particulates, there are several methods that can be used, including laser diffraction, optical microscopy, and/or SEM.

In certain aspects, the cores containing the active ingredient and lipid matrix have a flow point above 25° C., such as above 30° C., such as above 35° C., such as above 40° C.

In one aspect, the lipid matrix may contain fatty alcohols, fatty acids, fatty acid esters of glycerol, glycols and poly glycols, fatty acid esters of polyglycerol, polyglycolized glycerides, C8-C18 triglycerides, stearoyl polyoxylglycerides, lauroyl macrogol-32 glycerides, caprylocaproyl macrogol-8 glycerides, oleoyl macrogol-6 glycerides, linoleoyl macrogol-6 glycerides, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, propylene glycol monocaprylate esters, propylene glycol dicaprate/dicaprylate esters, propylene glycol heptanoate, propylene glycol monostearate, propylene glycol monooleate, propylene glycol monopalmitate, propylene glycol monomyristate, esterified alpha-tocopheryl polyethylene glycol succinate, propylene glycol monolaurate esters, polyoxyl 35 castor oil, polyoxyl 40 hydrogenated castor oil, lecithins, vitamin E, tocopheryl polyethylene glycol succinate (TPGS), sugar fatty acid esters, sorbitan fatty acid esters, polyoxyethylene sorbitan fatty acid esters, polyoxyethylene-polyoxypropylene copolymers, propylene glycol, triacetin, isopropyl myristate, diethylene glycol monoethyl ether, polyethylene glycol, glycerol, rosemary extract, ascorbic acid, ascorbyl palmitate, butylated hydroxyanisole (BHA), butylated hydroxytoluene (BHT), and mixtures and combinations thereof.

In a further aspect, the lipid matrix composition comprises greater than 50 wt % of the low flow point excipient. In one aspect, the lipid matrix composition comprises at least 2 wt % of the high flow point excipient. In yet a further aspect, the lipid matrix composition comprises less than 30 wt % of the high flow point excipient. Moreover, in one aspect, the mass ratio of the low flow excipient to the high flow excipient is at least 20:1, such as at least 15:1, such as at least 10:1, such as at least 4:1, such as at least 3:1, such as at least 2:1, or any ranges or values therebetween.

In another aspect, the lipid matrix composition contains greater than 50% by weight of one or more high flow point excipients. For example, in one embodiment, the lipid matrix is made exclusively from one or more high flow point excipients and does not contain a low flow point excipient. One or more high flow point excipients, for instance, can be present in the lipid matrix in an amount greater than about 40% by weight, such as an amount greater than about 50% by weight, such as in an amount greater than about 60% by weight, such as in an amount greater than about 65% by weight, such as in an amount greater than 70% by weight, and generally in an amount less than about 98% by weight, such as in an amount less than about 95% by weight, such as in an amount less than about 90% by weight, such as in an amount less than about 80% by weight, such as in an amount less than about 70% by weight. When greater amounts of high flow point excipients are present, one or more low flow point excipients may be present in the composition in an amount less than about 30% by weight, such as in an amount less than about 20% by weight, such as in an amount less than about 10% by weight and generally in an amount greater than 1% by weight, such as in an amount greater than about 4% by weight. The mass ratio of the high flow point excipients to the low flow point excipients can be from about 100:1 to about 1:1, such as from about 50:1 to about 10:1, such as from about 20:1 to about 5:1.

In one particular embodiment, the lipid matrix contains a wax combined with a fatty acid alcohol and a fatty acid. The wax, for instance, can comprise candelilla wax. The fatty alcohol, on the other hand, can be stearyl alcohol, while the fatty acid can be stearic acid. For example, the wax, such as candelilla wax, can be present in the composition in an amount greater than about 20% by weight, such as in an amount greater than about 25% by weight, and generally in an amount less than about 50% by weight, such as in an amount less than about 45% by weight. The fatty alcohol, on the other hand, can generally be present in an amount greater than about 10% by weight, such as in an amount greater than about 12% by weight, and generally in an amount less than about 25% by weight, such as in an amount less than about 22% by weight, such as in an amount less than about 18% by weight. The fatty acid, on the other hand, can be present in the composition in an amount greater than about 3% by weight, such as in an amount greater than about 5% by weight, such as in an amount greater than 7% by weight, and generally in an amount less than about 15% by weight, such as in an amount less than about 12% by weight, such as in an amount less than about 10% by weight.

In one aspect, the composition is contained within a lipid matrix containing stearyl alcohol, stearic acid, candelilla wax, and lecithin. In one aspect the lipid matrix may contain from about 1% about 80% by weight of the active ingredient, such as about 50% by weight of the active ingredient. Furthermore, in an aspect, the lipid matrix may contain from about 15% to about 25% by weight of stearyl alcohol, such as about 17% by weight of stearyl alcohol. Additionally or alternatively, in an aspect, the lipid matrix may contain from about 10% to about 20% by weight of stearic acid, such as from about 15% by weight of stearic acid. In one aspect, the lipid matrix may contain from about 10% to about 20% of a suitable wax, such as candelilla wax, such as from about 15% by weight of a suitable wax. Moreover, in one aspect, the lipid matrix may contain from about 1% to about 3% of a lecithin, such as about 2% of a lecithin, such as soy lecithin.

Nonetheless, it should be understood that lipid products made in accordance with the present disclosure, can be made very economically and can contain relatively large amounts of the one or more anti-inflammatory adjuvant(s). The composition of the present disclosure, for instance, can contain the one or more anti-inflammatory adjuvant(s) in the lipid matrix, in an amount greater than about 1% by weight, such as in an amount greater than about 5% by weight, such as in an amount greater than about 10% by weight, such as in an amount greater than about 15% by weight, such as in an amount greater than about 20% by weight, such as in an amount greater than about 25% by weight, such as in an amount greater than about 30% by weight. Moreover, in an aspect, the one or more anti-inflammatory adjuvant(s) can be present in the composition in an amount less than about 80% by weight, such as in an amount less than about 75% by weight, such as in an amount less than about 70% by weight, based on the total weight of the lipid multiparticulate particles containing anti-inflammatory adjuvant(s).

Examples of liquid matrices are described, for instance, in U.S. Patent Publication No. 2018/0125863, which is incorporated herein by reference. In one embodiment, the lipid matrix is different than forming micelles, microemulsions, macroemulsions, or liposomes.

In certain aspects, the lipid matrix containing the active ingredient may be formed and then formulated into one or more particles having a generally spherical shape and a mean diameter ranging from about ranging from 40 μm to 3000 μm, such as from about 100 μm to 2000 μm, such as from about 300 μm to 1000 μm.

In one aspect, the lipid matrix is comprised of at least 10 wt % to 50 wt % of the low flow point excipient. In another aspect, the lipid matrix is comprised of at least 50 wt % to 75 wt % of the low flow point excipient.

In one aspect, the lipid matrix comprises at least 2 wt % of the high flow point excipient. In another aspect, the lipid matrix is comprised of about 1 wt % to about 30 wt % of the high flow point excipient, such as about 2 wt % to about 20 wt % of the high flow point excipient, such as about 3 wt % to about 15 wt % of the high flow point excipient.

The lipid matrix may also include a dispersing agent. In one aspect, the lipid matrix includes from about 0 wt % to about 20 wt %, such as from about 0.01 wt % to about 20 wt %, of a dispersing agent. In another aspect, the lipid matrix includes from about 2 wt % to about 10 wt % of a dispersing agent.

The lipid matrix may also include an antioxidant. In one aspect, the lipid matrix includes from about 0 wt % to about 20 wt %, such as from about 0.01 wt % to about 20 wt %, of an antioxidant. In one aspect, the lipid matrix comprise from about 1 wt % to about 15 wt % of an antioxidant.

The lipid matrix may also contain flavoring or sweeteners to improve the taste of the particles to the user. In one embodiment, the lipid matrix comprise from 0 wt % to 15wt %, such as from 0.01 wt % to 10 wt %, of an flavoring or sweetener. In one embodiment, the lipid matrix comprise from 1 wt % to 5 wt % of an antioxidant flavoring or sweetener. Flavoring and sweeteners include essential oils other sweeteners used in the nutraceutical or food industries.

The lipid matrix may also include a flow aid. In one aspect, the lipid matrix includes from about 0 wt % to about 5 wt %, such as from about 0.01 wt % to about 5 wt %, of a flow aid. In another aspect, the lipid matrix includes from about 0.5 wt % to about 2 wt % of a flow aid.

The lipid matrix described herein may be formulated by any suitable process. In some embodiments, the matrix may be formulated by a suitable melt-spray-congeal process.

A molten mixture is formed by mixing and heating the lipid matrix compositions as previously described. Generally, the mixture is molten in the sense that it will flow when subjected to one or more forces such as pressure, shear, and centrifugal force, such as that exerted by a centrifugal or spinning-disk atomizer.

Once the molten mixture has been formed, it is delivered to an atomizer that breaks the molten mixture into small droplets. Virtually any method can be used to deliver the molten mixture to the atomizer. In certain embodiments of the disclosed methods the molten mixture is delivered to the atomizer by use of pumps and/or various types of pneumatic devices such as pressurized vessels or piston pots or extruder. In certain embodiments the molten mixture is maintained at an elevated temperature during delivery to the atomizer to prevent its solidification and to keep it in a flowable state.

When a centrifugal atomizer (also known as rotary atomizers or spinning-disk atomizer) is used, the molten mixture is fed onto a rotating surface, where it spreads outward and flows by centrifugal force. The rotating surface may take several forms, examples of which include a flat disk, a cup, a vanned disk, and a slotted wheel. The surface of the disk may also be heated to aid in atomization of the molten mixture or cooled to aid in the solidification of the cores containing the lipid matrix. Several mechanisms of atomization are observed with flat-disk and cup centrifugal atomizers, depending on the flow of molten mixture to the disk, the rotation speed of the disk, the diameter of the disk, the viscosity of the feed, and the surface tension and density of the feed. At low flow rates, the molten mixture spreads out across the surface of the disk and when it reaches the edge of the disk, forms a discrete droplet, which is then flung from the disk.

Once the molten mixture has been atomized, the droplets are congealed, typically by contact with a gas at a temperature below the solidification temperature of the composition. Typically, it is desirable that the droplets are congealed in less than 60 seconds, less than 10 seconds, or even in less than 1 second. In certain embodiments congealing at ambient temperature using an ambient temperature cooling medium, results in sufficiently rapid solidification of the droplets. However, as certain embodiments of the disclosed compositions are comprised of at least 50 wt % of a low flow point excipient, it is often preferred to utilize a cooling medium that is at a temperature that is at least 10° C. below ambient temperature. For some embodiments, it is preferred to utilize a cooling medium that is at least 20° C. below ambient temperature.

In one aspect, one or more surfactants can optionally be incorporated into the composition. Surfactants can be incorporated into the composition for various reasons. It was discovered that some surfactants can actually facilitate control of the delayed release function of the composition. In some embodiments, surfactants and co-surfactants may be included in the compositions. Exemplary surfactants and co-surfactants include polyethoxylated 12-hydroxysteric acid, also known as PEG15 hydroxy stearate (Kolliphor® HS-15), propylene glycol monocaprylate (C8) esters (Caproyl™ 90), esterified alpha-tocopheryl polyethylene glycol succinate (TPGS), mono, di, tricaprylic (C8) and capric acid (C10) esters of glycerol and mono and diesters of PEG400 (Labrasol®), Propylene glycol monolaurate (C12) esters (Labrafil® M1944CS), Polyoxyl 40 hydrogenated castor oil (Kolliphor® RH40), lecithins, and mixtures thereof.

In one embodiment, the surfactant incorporated into the composition can be a polysorbate, a sulfate surfactant, or mixtures thereof. Sulfate surfactants include, for instance, salts of fatty acids sulfates. For example, in one embodiment, the surfactant can be sodium laureth sulfate.

The amounts of surfactants incorporated into the composition can vary widely depending upon the reason for adding the surfactant or the desired result. In general, when included in the composition, one or more surfactants can be present in an amount greater than about 1% by weight, such as in an amount greater than about 3% by weight, such as in an amount greater than about 7% by weight, such as in an amount greater than about 10% by weight, such as in an amount greater than about 15% by weight, such as in an amount greater than about 20% by weight, such as in an amount greater than about 25% by weight, such as in an amount greater than about 30% by weight. One or more surfactants are generally present in the composition in an amount less than about 50% by weight, such as in an amount less than about 40% by weight, such as in an amount less than about 30% by weight, such as in an amount less than about 20% by weight, such as in an amount less than about 10% by weight.

Further advantages of encapsulating the anti-inflammatory adjuvant in the lipid multiparticulate include that the resulting lipid multiparticulates are stable and neutral tasting lipid. This is an advantage as anti-inflammatory adjuvants are typically odiferous which can result in some users rejecting anti-inflammatory adjuvants as nutraceuticals. In addition, the lipid multiparticulates of anti-inflammatory adjuvants, release the anti-inflammatory adjuvants, over a period of time once ingested. This will provide a lasting benefit of the anti-inflammatory adjuvant(s) providing an anti-inflammatory benefit to the use over a longer period of time, increased bioavailability in the GI tract, or a combination thereof. Further, the lipid multiparticulates may further increase the bioavailability of the anti-inflammatory adjuvant(s) as discussed above.

Other benefits of the lipid multiparticulates is that the anti-inflammatory adjuvant, can be in products such as nutritional bars; and in sachet formats for adding in to oatmeal, cereals, ready-to-mix (RTM) type beverages, salads, and other similar food products to achieve the benefits of the anti-inflammatory adjuvant.

In certain aspects, the cores disclosed herein may be subjected to additional processing in order to deposit one or more layers of an active ingredient material on the cores disclosed herein. For example, the cores described herein may contain one or more outer layers disposed on the core that include at least one active ingredient, which may be the same as one or more components in the composition or different from the components in the composition. Further, the one or more outer layers disposed on the core can include an adhesive in addition to one or more active ingredients. For example, to provide the outer layer disposed on the core, the core can be coated with suitable adhesives and then further coated with active ingredients in order to provide a particle having one or more outer layers including one or more active ingredients surrounding the core.

In certain aspects, the one or more particles may be subjected to any suitable coating process for edible tablets or particles in order to apply an active ingredient to the core or to the particles disclosed herein. Suitable processes may include those know generally as the Wurster process. Wurster processes are known in the art and may be synonymous with certain fluid bed microencapsulation processes. Descriptions of Wurster processes are disclosed in U.S. Pat. Nos. 2,648,609 and 3,241,520. Generally, during the Wurster process, the cores or particles are placed in a bed, such as a fluidized bed. The fluidized bed uses differential air flow to create a cyclic movement of the particle material. The one or more cores can be placed in the particulate bed and different air streams can move the bed of particulate material (i.e. the cores) as they are coated with a material, such as a suitable adhesive or active ingredient material, in order to coat the core structure. Generally, the process can be continued until the desired thickness of active ingredient layer is achieved on the core. The particles disclosed herein may be subjected to a suitable Wurster process in order to provide one or more active ingredient layers to the particle. In some embodiments, the cores or particles disclosed herein may be coated via any suitable fluid bed coating process.

In some aspects, the cores and/or particles may be coated via any known drum coating process. Generally, a drum coating process refers to a method for coating particles via placing the particles inside a rotating drum and applying the desired coating material to the particle while rotating the particles within the drum. Devices suitable for drum coating particles are known and include those described in U.S. patent publication no. 2015/0144058. In certain embodiments the cores disclosed herein may be placed in any suitable drum coating device and an adhesive can be applied to the cores or particles. Once sufficiently coated with the adhesive, an active ingredient can then be applied to the cores or particles in the drum to create an active ingredient layer on the cores or particles. This process of applying a suitable adhesive and active ingredient can be repeated as many times as necessary in order to produce the desired number of active ingredient layers on the particles. In certain embodiments, the adhesive may be applied to the core or particle directly with one or more active ingredients.

In one aspect, the cores or particles may be coated with a suitable adhesive. Suitable adhesives may include pharmaceutical grade shellacs such as pharmaceutical glaze, which is an alcohol-based solution that can include various types of food-grade shellac. In certain embodiment, the pharmaceutical glaze may contain from about 20% by weight to about 51% by weight of shellac in an ethyl alcohol solution. The pharmaceutical glaze may further contain additional additives such as waxes, titanium dioxide, and combinations thereof. In certain embodiments, the pharmaceutical glaze utilized according to embodiments herein is certified as generally recognized as safe (GRAS) by the U.S. Food and Drug Administration (FDA). In certain embodiments, the adhesive may include a suitable non-animal-based product, such as zein. Zein generally refers to a class of prolamin protein found in corn that can be manufactured as an adhesive coating or binder.

Accordingly, in some aspects, the outer surface of the cores described herein are coated with a sufficient amount of a suitable adhesive to enable binding of the active ingredient to the outer surface of the core. Generally, the adhesive can be applied such that outer surface of the core becomes tacky or sticky, but is not so tacky such that the cores or particles agglomerate together. Once the adhesive is applied, an active ingredient can be applied to the particle to create an active ingredient layer thereon. In some embodiments, the process of layering the particle with an adhesive layer and layering the particle with an active ingredient can be repeated as many times as desired in order to form a particle having a desired amount of active ingredient layers. For example, the particles disclosed herein may include a core having at least one outer layer thereon containing at least one active ingredient, such as at least two outer layers, such as at least three outer layers, such as at least four outer layers, such as at least five outer layers, such as at least six outer layers, etc. In certain embodiments, each of the outer layers may include one or more active ingredients. In some embodiments, one or more of the outer layers may include the same active ingredient or a different active ingredient from the active ingredient(s) contained within the core. In certain embodiments, the particle may contain one or more outer layers that do not contain an active ingredient. For example, the particle may contain one or more outer layers comprised of a suitable coating material for sealing the particle or for providing a particular release profile upon ingestion.

The joint health composition according to the present disclosure may be administered to the mammal including by oral, enteral or by-inhalation administration. However, in one aspect, it should be understood that the joint health composition is administered orally.

In some aspects, the one or more particles provided herein may be formulated into any suitable dosage formulation. For example, in certain embodiments, the one or more particles provided herein may be placed into a capsule for delivery by oral ingestion. Exemplary capsules include hard gelatin capsules, soft gelatin capsules, HPMC capsules, as well as capsules made from other materials. The one or more particles may be suspended in an aqueous-based matrix or an oil-based matrix within the capsule itself. In certain embodiments where the particles are suspended in an aqueous-based matrix or an oil-based matrix, the aqueous-based matrix or oil-based matrix may additionally include one or more active ingredients. In certain embodiments, the one or more particles may be contained within a monolithic enteric capsule suitable for providing a modified release profile when ingested.

Capsules normally include a shell filled with one or more specific substances. The shell itself may be a soft or a hard capsule shell. Hard capsule shells are generally manufactured using dip molding processes, which can be distinguished into two alternative procedures. In the first procedure, capsules are prepared by dipping stainless-steel mold pins into a solution of polymer, optionally containing one or more gelling agents (e.g. carrageenans) and co-gelling agents (e.g. inorganic cations). The mold pins are subsequently removed, inverted, and dried to form a film on the surface. The dried capsule films are then removed from the molds, cut to the desired length, and then the telescoping fit caps and bodies are assembled together, printed, and packaged. In the second procedure, no gelling agents or co-gelling agents are used and film-forming polymer solution gelification on the molding pins is thermally induced by dipping pre-heated molding pins into the polymer solution. This second process is commonly referred to as thermogellation, or thermogelling dip molding. The aforementioned manufacturing processes involve the use of solutions of the different ingredients that are needed for the making the telescoping fit hard capsule shells.

Hard capsules may be filled with active ingredients, such as the particles described herein, via procedures known in the art. Typically, active ingredients are combined with various compatible excipients for ease of fill. The resulting fill may be a dry powder, a granulation, particles, lipid particles, a suspension, or a liquid. Additionally, stable, filled hard capsules have advantages over other dosage delivery forms such as liquids and solid tablets. Certain active ingredients may be difficult to formulate into dry granules or may be otherwise incompatible with the tableting process. Another consideration is improved patient compliance for taste-masking and ease of swallowing, i.e., capsules being preferred by consumers over tablets. For example, in some embodiments, provided is a pharmaceutical composition that contains a capsule filled with the one or more particles disclosed herein. In some embodiments, the one or more particles have not been enterically coated for modified release or gastric protection.

In certain other embodiments, the one or more particles can be administered orally as a solid, liquid, suspension, or other suitable delivery means. The composition of particles may be administered via buccal or sublingual administration. In one embodiment, the one or more particles may be administered as a capsule, tablet, caplet, pill, troche, drop, lozenge, powder, granule, syrup, tea, drink, thin film, seed, paste, herb, botanical, and the like.

The joint health composition of the present disclosure may further comprise one or more excipients as further additives in the composition. Exemplary but non-limiting excipients and/or additives include antiadherents, such as magnesium stearate; binders, such as saccharides, sugar alcohols, gelatin, and synthetic polymers; coatings, such as cellulose ether hydroxypropyl methylcellulose (HPMC), shellac, corn protein zein, gelatin, fatty acids, fats, oils and/or waxes; coloring agents, such as titanium oxide and azo dyes; disintegrants, such as modified starch sodium starch glycolate and crosslinked polymers including polyvinylpyrrolidone and sodium carboxymethyl cellulose; fillers, such as maltodextrin; flavoring agents, such as mint, liquorice, anise, vanilla, and fruit flavors including peach, banana, grape, strawberry, blueberry, raspberry, and mixed berry; glidants, such as fumed silica, talc, and magnesium carbonate; lubricants, such as talc, silica, and fats including vegetable stearin, magnesium stearate, and stearic acid; preservatives, such as antioxidants, vitamins, retinyl palmitate, selenium, the amino acids cysteine and methionine, citric acid, sodium citrate, and parabens; sorbents; sweeteners, such as sucrose and sucralose; and vehicles, such as petrolatum and mineral oil.

In one aspect, the joint health composition of the present disclosure may be combined with various additives and components that can improve one or more properties of the composition. For example, in one embodiment, the additive composition may be combined with a stabilizer package that may serve to stabilize at least one property of the composition. In one particular embodiment, for instance, a stabilizer package may be added to the composition in an amount sufficient to reduce the hydroscopic properties of the composition and/or prevent the composition from absorbing moisture. A stabilizer package may also be combined with the composition in order to improve the handling properties of the composition. For instance, the stabilizer package may allow the composition to have better flow properties, especially when in granular form.

In one aspect, the joint health composition may be combined with a polymer binder in conjunction with a stabilizer package. In addition, a coating material may also be applied to the composition after the composition has been combined with the polymer binder and the stabilizer package. The coating material, for instance, may contain at least one fat. In accordance with the present disclosure, the above components can be added to any suitable pharmaceutical composition in addition to the composition of the present disclosure. For instance, the above components may be added to any pharmaceutical composition containing a carnitine or an amino acid.

The polymer binder and the stabilizer package may be combined with the joint-health composition in a manner that homogeneously incorporates the stabilizer package into the product. In one embodiment, for instance, the composition of the present disclosure is first combined with a polymer binder, such as through a spray dry process, and then combined with the stabilizer package. The polymer binder may comprise any suitable pharmaceutically acceptable polymer, such as film-forming polymers and/or polysaccharides. Particular examples of polymer binders that may be used in accordance with the present disclosure include starch, maltodextrin, gum arabic, arabinogalactan, gelatin, and mixtures thereof. In one embodiment, the polymer binder is added to the pharmaceutical composition in an amount of at least about 5% by weight, such as at least about 8% by weight, such as at least about 10% by weight, such as at least about 15% by weight. One or more polymer binders are present in the composition in an amount less than about 50% by weight, such as in an amount less than about 45% by weight, such as in an amount less than about 40% by weight, such as in an amount less than about 35% by weight, such as in an amount less than about 30% by weight.

In one embodiment, the polymer binder may comprise a starch, such as a modified starch. The starch, for instance, may be derived from corn or waxy maize. In one embodiment, the starch may comprise HI-CAP100 starch sold by National Starch and Chemical Company. In an alternative embodiment, the polymer binder may comprise arabinogalactan.

Once the polymer binder is combined with the composition such as through a spray dry process, the resulting mixture can then be combined with a stabilizer package. In one embodiment, the stabilizer package comprises oxide particles in combination with a salt of a carboxylic acid. In one particular embodiment, the stabilizer package may comprise a dry product, such as a powder or granular product that is combined with the composition and polymer binder. The combination of oxide particles and a salt of a carboxylic acid have been found to provide numerous advantages and benefits when combined with the composition. For instance, the stabilizer package has been found to stabilize the composition and make the composition less hydroscopic. The composition is also easier to handle and, when in granular form, produces a free-flowing product.

The oxide particles that may be added to the supplement composition may comprise silica. For instance, the oxide particles may comprise precipitated silica particles. The silica particles may have a particle size (d50, laser diffraction following ISO Test 13320) of less than about 55 microns, such as less than about 40 microns, such as less than about 30 microns, such as less than about 25 microns, such as less than about 20 microns, such as less than about 15 microns, such as less than about 12 microns, such as less than about 10 microns, such as less than about 8 microns, such as less than about 6 microns, such as less than about 4 microns, such as less than about 2 microns, such as less than about 1 micron. The particle size is typically greater than about 0.5 microns, such as greater than about 1 micron. The particles may have a specific surface area (ISO Test 9277) of greater than about 120 m2/g, such as greater than about 130 m2/g, such as greater than about 150 m2/g, such as greater than about 170 m2/g, such as greater than about 200 m2/g, such as greater than about 220 m2/g. The specific surface area is generally less than about 500 m2/g. The oxide particles, such as the silica particles, can be present in the pharmaceutical composition in an amount greater than about 0.01% by weight, such as in an amount greater than about 0.05% by weight, such as in an amount greater than about 0.1% by weight. The oxide particles are generally present in an amount less than 5% by weight, such as in an amount less than about 2% by weight, such as in an amount less than about 1.5% by weight, such as in an amount less than 0.5% by weight.

In addition to the oxide particles, the stabilizer package may also include a salt of a carboxylic acid. The salt of a carboxylic acid may comprise a salt of a fatty acid. The fatty acid, for instance, may have a carbon chain length of from about 6 carbon atoms to about 40 carbon atoms, such as from about 12 carbon atoms to about 28 carbon atoms. In one embodiment, the salt of the carboxylic acid may comprise a stearate salt. The stearate salts that may be used include calcium stearate, sodium stearate, magnesium stearate, mixtures thereof, and the like. In one embodiment, the salts of the carboxylic acid may include both hydrophilic groups and hydrophobic groups. The salt of the carboxylic acid may be present in the composition in an amount greater than about 0.5% by weight, such as in an amount greater than about 1% by weight, such as in an amount greater than about 1.5% by weight. The salt of the carboxylic acid is generally present in an amount less than about 5% by weight, such as in an amount less than about 4% by weight, such as in an amount less than about 3% by weight.

In addition to the polymer binder and the stabilizer package, the composition may include various other components and ingredients. In one embodiment, for instance, the composition may contain a citric acid ester, such as a citric acid ester of a mono and/or diglyceride of a fatty acid. The composition may also contain a lecithin, such as a lecithin obtained from rapeseed, sunflower, and the like. The above components can be present in the composition in relatively minor amounts, such as less than about 2% by weight, such as less than about 1.5% by weight, such as less than about 1% by weight. The above components are generally present in an amount greater than about 0.05% by weight, such as in an amount greater than about 0.1% by weight. Moreover, as discussed above, it should be understood that, in one aspect, the joint-health composition may include one or more additional active ingredients, optionally contained in the LMP.

Furthermore, in one aspect, the joint-health composition may be formulated into a food and/or supplement for exercise, sport, or daily nutritional purposes. In such an aspect, the joint-health composition may further include at least one vitamin, such as at least one of vitamin B, vitamin C, and vitamin E. Vitamins may be contained in the supplement in an amount of from about 50 μg/g of supplement to about 5000 μg/g, such as about 100 μg/g to about 4500, such as about 250 μg/g to about 4000 μg/g, such as about 400 μg/g to about 3500 μg/g, or any ranges or values therebetween. The above ranges may be for any one vitamin alone or a total amount of all vitamins. In one aspect, vitamin E is present in supplement in an amount of about 100 μg/g to about 1000 μg/g, such as about 250 μg/g to about 750 μg/g, such as about 400 μg/g to about 600 μg/g, or any ranges or values therebetween. In another aspect, vitamin C is present in supplement in an amount of about 1000 μg/g to about 5000 μg/g, such as about 2000 μg/g to about 4000 μg/g, such as about 3000 μg/g to about 3750 μg/g, or any ranges or values therebetween.

Furthermore, in an aspect, the joint-health composition contains at least one mineral, such as at least one of potassium magnesium, zinc, or calcium. Minerals may be contained in the joint-health composition in an amount of from about 1 mg/g to about 50 mg/g, such as about 2.5 mg/g to about 45 mg/g, such as about 5 mg/g to about 40 mg/g, or any ranges or values therebetween. The above ranges may be for any one mineral or a total amount of one mineral. In one aspect, the joint-health composition contains potassium in an amount of about 9.5 mg/g to about 12 mg/g, such as about 9.75 mg/g to about 11.5 mg/g, such as about 10 mg/g to about 11 mg/g, or any ranges or values therebetween. Similarly, in one aspect, the joint-health composition contains magnesium in an amount of about 1 mg/g to about 10 mg/g, such as about 2.5 mg/g to about 7.5 mg/g, such as about 4 mg/g to about 6 mg/g, or any ranges or values therebetween. Furthermore, in one aspect, the joint-health composition contains calcium in an amount of about 1 mg/g to about 50 mg/g, such as about 2.5 mg/g to about 47.5 mg/g, such as about 5 mg/g to about 45 mg/g, such as about 10 mg/g to ab out 40 mg/g, such as about 20 mg/g to about 37.5 mg/g, such as about 30 mg/g to about 35 mg/g, or any ranges or values therebetween.

Thus, in one aspect, the joint-health composition according to the present disclosure can be combined with or used with other nutraceutical components to form a nutraceutical composition, such as the joint-health composition described herein. The lipid multiparticulates of anti-inflammatory adjuvant(s), can be blended with other nutraceutical components which result in stable combinations of lipid multiparticulates of anti-inflammatory adjuvants(s), collagen, and other nutraceutical ingredients in both nutraceutical finished solid and liquid dosages, as well as in food and beverage applications. Exemplary nutraceuticals which can be blended with the lipid multiparticulates and collagen include probiotics, for example, but not limited to TWK10® product available from Lonza, enzymes, endogenous fatty acid amides, cetylated fatty acid esters, omega-3 fatty acids, hyaluronic acids, curcuminoids, herbal and botanical extracts, carotenoids, methylsulfonylmethane (MSM), carnitine, including but not limited to, Carnipure® available from Lonza, and antioxidants, for example, Oceanix™ available from Lonza. Other nutraceutical ingredients having anti-inflammatory benefits such as turmeric curcuminoids, eggshell membrane, green lipped mussel, omegas-3 EPA and DHA, krill oil, French maritime pine bark extract (Pycnogenol®), Scutellaria baicalensis and Acacia catechu extracts (Univestin®), ashwagandha extract, rose hip extract, tart cherry extract, astaxanthin, hops extract (Perluxan®), glucosamine, chondroitin, hyaluronic acid, salmon nasal cartilage, avocado soy unsaponifiable, methylsulfonylmethane (MSM), willow bark extract, tamarind seed extract, lactobacillus and bifidobacteria probiotic strains (e.g. TWK10® product available from Lonza), palmitoylethanolamide (PEA), and cetyl myristoleate (CM), which may further eliciting anti-inflammation health benefits.

Moreover, the joint-health composition may be suitable for administration to any mammal. For instance, the mammal may be human or canine. The joint-health composition can be fed to a mammal of any age such as from parturition through the adult life in the mammal. In various embodiments the mammal may be a human, dog, a cat, a horse, a pig, a sheep, or a cow. In many embodiments, the mammal can be in early to late adulthood.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A joint health composition for improving one or more of joint health and inflammation, comprising
a lipid multiparticulate comprising at least one anti-inflammatory adjuvant and a lipid matrix, wherein the anti-inflammatory adjuvant is dispersed within the lipid matrix, and
collagen;
wherein the anti-inflammatory adjuvant comprises an extract or isolate from a natural source that inhibits one or more of: interleukin-1 expression, interleukin-6 expression, NF-KB expression, cyclooxygenase-2 (COX-2) expression, inducible nitric oxide synthase (iNOS) expression, 5-lipoxygenase biosynthesis, and tumor necrosis factor-α (TNF-α) expression.

2. The composition according to claim 1, wherein the anti-inflammatory adjuvant is encapsulated by the lipid matrix.

3. The composition according to claim 1, wherein the anti-inflammatory adjuvant is present in the lipid multiparticulate in an amount from about 1% to about 80% by weight based on a total weight of the lipid multiparticulate particles.

4. The composition as defined in claim 1, wherein the joint-health composition is in a form of a capsule, a tablet, a powder, or is suspended in a liquid.

5. The composition as defined in claim 1, wherein collagen is contained in the joint health composition in an amount greater than about 50 mg up to about 200 mg or less.

6. The composition as defined in claim 1, wherein the anti-inflammatory adjuvant is contained in the joint health composition in an amount from about 2.5 mg to about 200 mg.

7. The composition as defined in claim 1, wherein the collagen comprises undenatured Type II collagen or native type II collagen.

8. The composition as defined in claim 1, wherein the anti-inflammatory adjuvant comprises an extract and/or isolate obtained from Andrographis paniculate, Tamarind seed, Boswellia serrata, green lipped mussel (Perna canaliculus), turmeric (Curcuma longa), stinging nettle (Urtica dioica), Cat's claw (Uncaria tomentosa), or bromelain.

9. The composition as defined in claim 1, wherein the anti-inflammatory adjuvant is a boswellic acid, tamarind seed extract, an andrographolide, or combinations thereof.

10. The composition as defined in claim 1, wherein the anti-inflammatory adjuvant is
(a) an andrographolide, and wherein the andrographolide is contained in the joint health composition in an amount from about 2.5 mg to about 200 mg,
(b) a tamarind seed extract, and wherein the tamarind seed extract is contained in the joint health composition in an amount from about 10 mg to about 200 mg, or
(c) a boswellic acid, wherein the boswellic acid comprises 11-keto-β-boswellic acid, acetyl-11-keto-β-boswellic acid, or a combination thereof, and wherein the boswellic acid is contained in the joint health composition in an amount from about 10 mg to about 200 mg.

11. (canceled)

12. (canceled)

13. (canceled)

14. A method of improving one or more of joint pain and inflammation in a mammal, comprising:
supplying to the mammal a therapeutically effective amount of a joint-health composition comprising collagen and at least one anti-inflammatory adjuvant, wherein the at least one anti-inflammatory adjuvant is in the form of a lipid multiparticulate; and
wherein the anti-inflammatory adjuvant comprises an extract or isolate from a natural source that inhibits one or more of: interleukin-1 expression, interleukin-6 expression, NF-kB expression, cyclooxygenase-2 (COX-2) expression, inducible nitric oxide synthase (iNOS) expression, 5-lipoxygenase biosynthesis, and tumor necrosis factor-α (TNF-α) expression.

15. The method as defined in claim 14, wherein the improvement in one or more of joint pain and inflammation is evidenced by a 10% or greater decrease in serum interleukin-1, interleukin-6, TNF-α, COX-2, SOX-9, MMP-13, or a combination thereof, or a subjective reduction in one or more of joint pain and inflammation, in about two weeks or less.

16. The method as defined in claim 14 wherein the therapeutically effective amount of the joint health composition administered to the mammal includes a collagen amount of at least 0.3 mg/kg of body weight per day to about 3 mg/kg of body weight per day.

17. The method as defined in claim 14, wherein the collagen comprises undenatured Type II collagen or native type II collagen.

18. The method as defined in claim 14, wherein the anti-inflammatory adjuvant comprises an extract and/or isolate obtained from Andrographis paniculate, Tamarind seed, Boswellia serrata, green lipped mussel (Perna canaliculus), turmeric (Curcuma longa), stinging nettle (Urtica dioica), Cat's claw (Uncaria tomentosa), or bromelain.

19. The method as defined in claim 14, wherein the anti-inflammatory adjuvant is a boswellic acid tamarind seed extract (TSE), an andrographolide, or combinations or derivatives thereof.

20. The method as defined in claim 14, wherein the improvement in one or more of joint pain and inflammation is evidenced in about one week or less.

21. The method as defined in claim 14, wherein the improvement in one or more of joint pain and inflammation is evidenced by a decrease in serum: interleukin-1, interleukin-6, TNF-α, COX-2, SOX-9, MMP-13, or a combination thereof of at least about 15%, after 10 days or less.

22. The method as defined in claim 14, wherein improvement in one or more of joint pain and inflammation is evidenced by a decrease in
(a) interleukin-1 serum of at least about 20%,
(b) interleukin-6 serum of at least about 20%, or
(c) matrix metallopeptidase 13 serum of at least about 20%.

23. (canceled)

24. (canceled)

25. The method as defined in claim 14, wherein the joint health composition is administered to the mammal in a therapeutically effective amount sufficient to change a Kellgren-Lawrence score of the mammal by at least about 20%.

26. The method as defined in claim 14, wherein the joint health composition administered daily.

27. A joint health composition for improving one or more of joint health and inflammation, comprising
an extract and/or isolate obtained from Andrographis paniculate, Tamarind seed, green lipped mussel (Perna canaliculus), turmeric (Curcuma longa), stinging nettle (Urtica dioica), Cat's claw (Uncaria tomentosa), and bromelain; and
collagen.

28. The joint health composition as defined in claim 27, wherein the extract and/or isolate is from Andrographis paniculate; or Tamarind seed, and the collagen is undenatured Type II collagen.
